(19) United States
(12) Patent Application Publication
(10) Pub. No.: US 2024/0408334 A1
(43) Pub. Date: Dec. 12, 2024

(54) METHOD, APPARATUS, AND SYSTEM FOR DETERMINING PHYSIOLOGICAL EFFECTIVENESS OF AN AIRWAY PRESSURE DEVICE

(71) Applicant: NOX MEDICAL, Reykjavik (IS)

(72) Inventors: Alp Sinan BARAN, Reykjavik (IS); Sveinbjorn HOSKULDSSON, Reykjavik (IS); Jon Skirnir AGUSTSSON, Reykjavik (IS)

(21) Appl. No.: 18/738,327

(22) Filed: Jun. 10, 2024

Related U.S. Application Data
(60) Provisional application No. 63/507,034, filed on Jun. 8, 2023.

(51) Int. Cl. A61M 16/00; G16H 20/40

(52) U.S. Cl. CPC A61M 16/026; A61M 16/0003; G16H 20/40; A61M 2205/3327; A61M 2205/52

(57) ABSTRACT

Methods, apparatuses, and systems for determining a physiologically effective use of a positive airway pressure device of a target subject. The method includes obtaining one or more parameters recorded by the positive airway pressure device and determining a range of values for the one or more parameters. A simulated or ineffective use of the positive airway pressure device is identified when the range of values is outside a predefined tolerance range and a physiologic use of the positive airway pressure device is identified when the range of values is inside the predefined tolerance range.

### CROSS-REFERENCE TO RELATED APPLICATIONS

This application claims the benefit of and priority to U.S. Provisional Patent Application Ser. No. 63/507,034 filed on Jun. 8, 2023, which is expressly incorporated herein by reference.

### FIELD OF THE DISCLOSURE

The present disclosure relates to a method, apparatus, and system for determining whether an airway pressure device is being used by a subject, whether an airway pressure device is being effectively used, and/or whether the subject using the an airway pressure device has an abnormal medical condition. The disclosure can be used as chain of custody or means of verification of the veracity of signals obtained in a physiological study or treatment. It can be used to determine whether a signal originating from an unknown source, single person, or subject of a physiological study or treatment corresponds to known physiologic respiratory parameters during use of an airway pressure device by a known source, person, or subject of the physiological study or treatment, and thus can be used to determine whether an airway pressure device of a physiological study or treatment assigned to a person or study subject was indeed placed on the person or study subject, was correctly placed, and the recorded physiologic respiratory data were indeed within physiologic respiratory parameters.

### RELATED ART

The objective of this disclosure is to determine whether provided signals originate from, or in other words correspond to, a desired source, person, or subject and represent actual use as intended of an airway pressure device.

Many physiological studies or treatments rely on accurate and effective use of a therapeutic device by a subject. Such therapeutic devices are often unwieldy, complex, or otherwise difficult to correctly employ. While a subject may be instructed according to appropriate use of the therapeutic device, the inventors of the present disclosure have identified a significant need to identify and classify actual use as intended of a therapeutic device by measuring one or more signals using a single or multiple sensors.

The inventors of the present application have found that the use of the therapeutic device is currently assumed to be by the subject to whom it was prescribed, without sufficient scrutiny of the physiologic data recorded by the device to validate actual use, which remains a matter of subjective and sometimes arbitrary intuition, preferences, and user experience, rather than being an exercise in quantitative precision. The use and monitoring of a therapeutic device may thus be subject to numerous errors and inefficiencies, as well as intended deception to mimic actual use. Moreover, the inventors of the present application have found that a more thorough analysis of the physiologic respiratory parameters during use of an airway pressure device may help in identifying individual subjects with an underlying, abnormal medical condition.

### SUMMARY

The present disclosure concerns a method, system, and apparatus for determining actual physiologic use of a positive airway pressure machine of a target subject (“physiologic use”), an effective use of a positive airway pressure machine by the subject, or an abnormal condition of the subject. The method includes obtaining one or more parameters recorded by the positive airway pressure machine. Deceptive or simulated use (“simulated use”) of the positive airway pressure device, ineffective use or misuse, or an abnormal medical condition may be identified when a deviation value is determined by one or more of the recorded parameters being outside known physiologic ranges and physiologic use of the positive airway pressure device by a subject is identified when the recorded values are all inside the known range for each value.

Additionally, a system configured to determine a physiologic use of a positive airway pressure machine is provided. The system comprises one or more processors and one or more memory storages. The one or more memory storages have stored thereon one or more parameters recorded by the positive airway pressure machine. The one or more processors are configured to perform the following: determine the range of values of the one or more parameters; identify nonphysiologic use when one or more of the recorded parameters is outside known human physiologic ranges, and identify physiologic use when recorded values are all inside the known human ranges for each value.

And one or more computer-readable mediums are provided herein having stored thereon executable instructions that when executed by the one or more processors configure a computer system to perform at least the following: obtain one or more parameters recorded by a positive airway pressure machine; determine a range of values of the one or more parameters; identify nonphysiologic use when one or more of the recorded parameters is outside known human physiologic ranges, and identify physiologic use when recorded values are all inside the known human ranges for each value.

### BRIEF DESCRIPTION OF THE DRAWINGS

FIG. 1 shows a diagram of a positive airway pressure (PAP) device as may be used according to embodiments of the current disclosure.

FIG. 2 shows a diagram of a system for performing a method according to embodiments of the current disclosure.

FIG. 3 shows flow diagram of a method according to embodiments of the current disclosure.

FIG. 4 shows a one-night plot of pressure parameters recorded by a PAP device for non-human simulated breathing and for physiologic human breathing.

FIG. 5A shows a one-night plot of parameters recorded by a PAP device for a target subject employing a simulated breathing device.

FIG. 5B shows a one-night plot with simulated breathing in the first half and physiologic human breathing in the second half.

FIG. 6 shows a daily plot of parameters recorded by a PAP device for another target subject employing a simulated breathing device.

FIG. 7 shows a one-night and daily plots of parameters recorded by a PAP device for another target subject employing a simulated breathing device. The one-night plot is from the date indicated with the red arrow on the daily plot.

### DETAILED DESCRIPTION OF VARIOUS EMBODIMENTS

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings and are described below. The connections and arrangements represented in the figures introduced above are to be understood as exemplary and are not necessarily shown in proportion or in exhaustive detail. It should be understood, however, there is no intention to limit the disclosure to the specific embodiments disclosed, but on the contrary, the intention covers all modifications, alternative constructions, combinations, and equivalents falling within the spirit and scope of the disclosure.

The flowchart illustrations and block diagrams in the flow diagrams illustrate the architecture, functionality, and operation of possible implementations of systems, methods, and computer program products according to various embodiments of the present disclosure. In this regard, each block in the flowchart illustrations or block diagrams may represent a module, segment, or portion of code, which comprises one or more executable instructions for implementing the specified logical function(s).

It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable media that can direct a computer or other programmable data processing apparatus to function in a particular manner, such that the instructions stored in the computer-readable media produce an article of manufacture including instruction means which implement the function/act specified in the flowchart illustrations and/or block diagram block or blocks.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present disclosure, the only relevant components of the device are A and B.

Reference throughout this specification to “one embodiment” or “an embodiment” means that a particular feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment but may. Furthermore, the particular features, structures or characteristics may be combined in any suitable manner into one or more embodiments.

Similarly, it should be appreciated that in the description of exemplary embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the embodiments require more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

Furthermore, while some embodiments described herein include some, but not other features included in other embodiments, combinations of features of different embodiments are meant to be within the scope of the disclosure, and form different embodiments. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description. Examples of the current disclosure may be provided in terms referring to respiratory rate, minute ventilation, tidal volume, inspiratory time, pressure, inspiratory pressure, peak inspiratory pressure, and expiratory pressure, such as including a classification of a measurement as an artifact, a potential problem or as having no apparent evidence of an abnormality, however there is no intention to limit the disclosure thereto. The same or similar principles of embodiments of the current disclosure may be applied to alternative and additional physiological parameters and classifications.

A primary problem being resolved by this disclosure is determining whether an airway pressure device was physiologically used as intended by a subject, such as by determining whether provided signals originate from, or in other words correspond to, a desired source, person, or patient.

Many physiological studies or treatments rely on accurate and effective use of a therapeutic device by a subject. Such therapeutic devices are often unwieldy, complex, or otherwise difficult to correctly employ. While a subject may be instructed according to appropriate use of the therapeutic device, the inventors of the present disclosure have identified a significant need to identify and classify appropriate physiologic use of a therapeutic device by measuring one or more signals using a single or multiple sensors to determine whether it was used by a breathing subject or an alternate method of generating cyclic airflow to simulate, for example, human breathing.

The sensors used to monitor the signals may be a part of one system or device, or be a compilation of several systems or devices, where sensors from a single or different manufacturers are used together to obtain the measurement. The sensors used can be medical or consumer products or a combination of the two. The sensor can communicate over a wire or wirelessly, or their signals can be collected by a third system.

The high cost of manual evaluation of one or more signals may limit the effectiveness and availability of therapeutic care. Further, the manual evaluation of such signals inevitably results in inaccuracies. With stringent medical safety and diagnostic requirements that increasingly require levels of accuracy and consistency achievable only by automated systems, rather than by human evaluation, the inventors have identified a need for a method, apparatus, and system for automatically and accurately identifying and classifying physiologic and nonphysiologic (simulated) use of a therapeutic device. Further, the inventors have identified a need for a method, apparatus, and system for automatically and accurately identifying and classifying effective physiologic use of a therapeutic device. Lastly, the inventors have identified a need for a method, apparatus, and system for automatically and accurately identifying subjects using therapeutic device having an underlying abnormal medical condition.

Unfortunately, the difficulty of this task is compounded by the fact that a person or subject may intentionally “cheat” to give the appearance of treatment compliance. That is, as described below, some subjects may be incentivized to purposely provide signals obtained from two different subjects, obtained from a mechanical device, or manipulated after collection, or in other words “cheat.” It would be helpful to be able to verify, or in other words, determine the veracity of use in such treatments.

For example, some sleep studies or treatments are mandatory and are required to regulate the physical and mental capabilities of operators working in hazardous environments or with risky machinery or tools. This can be, for example, a truck driver that needs to prove that he is capable of driving a truck without risking the lives of others by inattentiveness or falling asleep, etc. Various governmental agencies, such as departments of transportation (DOTs), insurance companies, or employers may desire that the driver prove that he does not have an untreated sleep disorder or is sufficiently compliant with necessary treatment for a sleep disorder for him to operate the truck.

On the other hand, providers such as health insurance companies or the U.S. Department of Veterans Affairs (VA) may encounter a problem where patients receive extra benefits related to use of a therapeutic device, but the device is not used in a physiologic manner.

For these cases, it is important to determine that the treatment was physiologically used so that the usage time legitimately counts toward the required hours of use necessary to be considered compliant with the treatment for the person who was supposed to receive treatment. For example, it is important to determine that a positive airway pressure (PAP) device was appropriately worn and used by a target subject for a predetermined time and not removed, whether unintentionally or deceptively, such as accidentally knocked out of place during sleep or intentionally connected to a cheat device. Nonphysiologic use should be rejected and not counted toward the required duration of therapeutic use.

The identification and classification of physiologic and nonphysiologic use, or effective or ineffective use of a therapeutic device remains a matter of subjective and sometimes arbitrary intuition, preferences, and user experience, rather than being an exercise in quantitative precision. The use and monitoring of a therapeutic device may thus be subject to numerous errors and inefficiencies. Currently, nonphysiologic use by connecting a PAP machine to a device that generates cyclic airflow can go undetected and can count toward therapeutic usage time of the CPAP machine, despite one or more monitored parameters being clearly outside the limits of human physiology.

The present disclosure addresses and solves the above-noted problems in a new way. Particularly, by evaluating one or more parameters from signals obtained during use of a therapeutic device.

The signals may be physiologic signals, nonphysiologic signals intended to simulate human use, and/or, one or more parameters, or aggregate parameters thereof. One or more parameters, or aggregate parameters of interest may be obtained from signals collected by a device or another sensor, and/or the signals collected by the device or another sensor may comprise the aggregate parameters of interest. The signals may be recorded by any body-worn or therapeutic device, such as a mobile device, smart watch, activity tracker, health, or medical sensors. One particular example includes signals obtained by a positive airway pressure device that records multiple signals coming from one or more independent sensors. Such a positive airway pressure device could be, for example, a ResMed AirSense Positive Airway Pressure (PAP) machine from ResMed Inc.; an XT Auto CPAP machine by Apex Medical; a Luna (2) Auto CPAP machine; a Transcend Auto CPAP; a DreamStation CPAP Machine; IntelliPap AutoAdjust Auto CPAP Machine

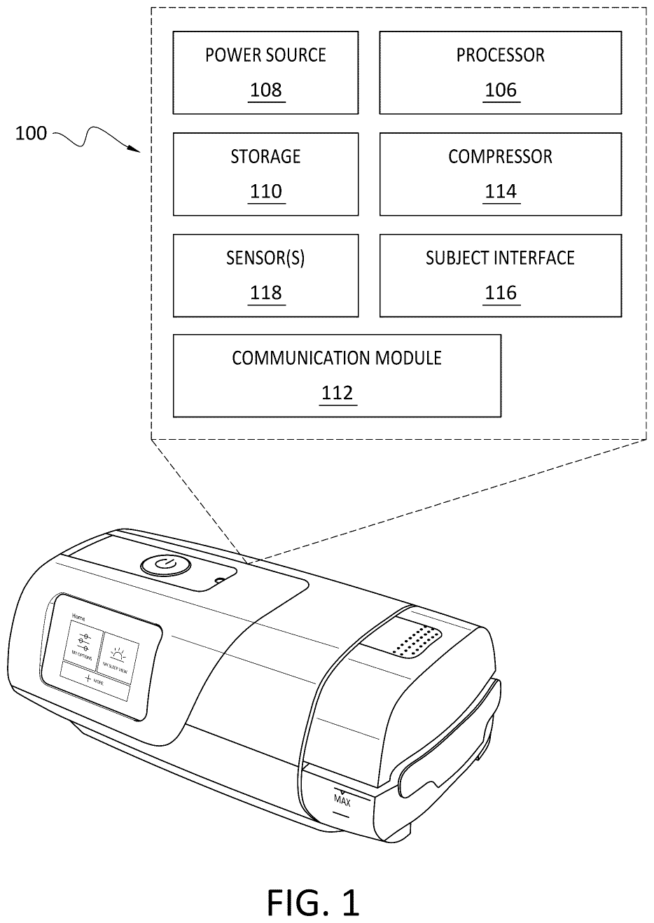

FIG. 1 shows a schematic of a PAP device 100, such as may be provided for treating sleep apnea by regulating airway pressure of a subject. The PAP device may include a compressor 114, a subject interface 116 for connecting to a subject's nose and/or mouth, and one or more sensors 118 configured to collect signals related to the subject. In operation, the PAP device 100 may be arranged to collect measurements from physiologic signals of a body of a target subject using the one or more sensors 118, such that the collected measurements may be stored in a memory 110 and/or communicated to another computing device using a communication interface 112. The one or more sensors 118 may include any suitable sensor that may be arranged for monitoring physiologic signals from the body of the subject or the like.

For example, the one or more sensors 118 may include pressure sensors, differential pressure sensors, temperature sensors, humidity sensors and the like, and may be configured for collecting signals corresponding to parameters of the subject's breathing, such as respiratory rate, tidal volume, minute ventilation, inspiratory time, pressure, inspiratory pressure, peak inspiratory pressure, expiratory time, and expiratory pressure. The device or the sensors thereof may obtain signals that may originate from several sources and/or may provide the obtained signals to another processing device that obtains signals that may originate from several sources.

The PAP device 100 may include a processor 106, a power source 108, and a memory 110 for storing and evaluating the signals collected by the one or more sensors 118. Data between components of the PAP device may be transmitted via wireless communication, such as Bluetooth, cellular signals or the like, or via wired communication. The processor 106 may be configured to control and regulate operation of the compressor 114 of the device 100, for example by adjusting an applied pressure according to resistance in the subject's breathing or by detecting leakage, such as from an improperly placed mask. However, there is currently no approach for determining whether the signals collected by the device 100 are authentic and represent physiologic use of the device 100 by a subject, such as a human subject, and nonphysiologic use is not identified so that it can be deducted from therapeutic usage time monitored to determine treatment compliance.

In a preferred embodiment, the data used in the evaluation and determination of physiologic use or nonphysiologic use, effective or ineffective use by the subject, or to identify an abnormal medical condition of the patient is based on sensors or parameters recorded or obtained by a standard PAP device. However, according to other embodiments, the method and system described herein may be based on any combination of additional body-worn devices or sensors, non-medical or medical, and/or one or more further computing devices. For example, a PAP device could be used alone, or in combination with another device, for example, Nox Medical's T3 device or another similar sleep study device, and/or one or more further computing devices. A sleep study device may include one or more sensors as with the PAP device, such as sensors for collecting different physiological signals relative to the PAP device and/or redundant sensors for collecting the same physiological signals. Accordingly, the PAP device may include a communication module 112 arranged for communicating with such devices.

A computing device may be provided for identifying a physiologic use of the PAP device according to embodiments of the disclosure, or effective or ineffective e use of the PAP device according to embodiments of the disclosure. A system for determining a physiologic use of the PAP device may include one or more of a PAP device and a computing device, with or without additional sensors and/or devices.

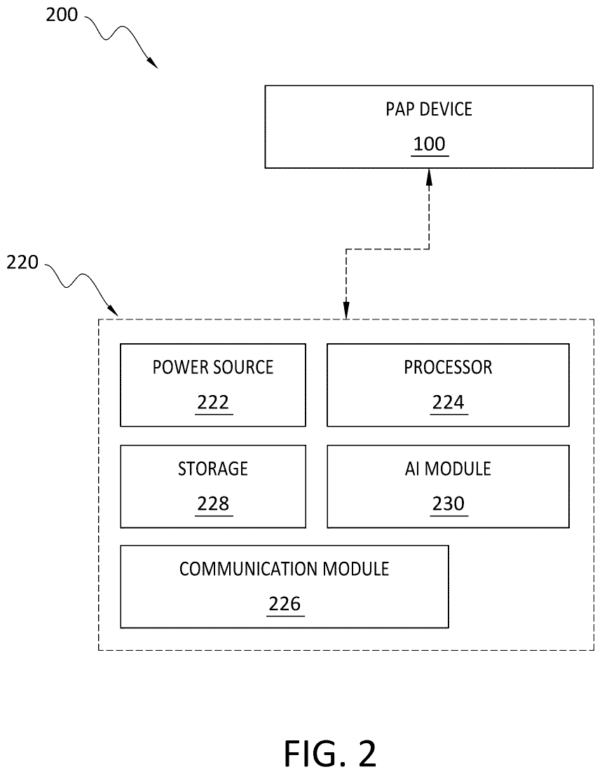

FIG. 2 is a diagram of a system 200 including a computing device 220 for identifying physiologic use of a PAP device 100 according to an embodiment of the present disclosure. The computing device 220 may comprise a power source 222, a processor 224, a communication module 226, and a storage 228. In varying examples, the computing device 220 may be a mobile computing device, such as a mobile phone or a laptop computer, a remote server device or the like.

The storage 228 may comprise instructions for operating a system for identifying physiologic use of a PAP device 100 stored thereon in a non-transitory form that, when executed by the processor 224, cause the processor 224 to carry out one or more of the steps described herein, in particular obtaining one or more parameters collected by the PAP device 100, determining any values outside of physiologic ranges for one or more parameters, which will be referred to herein as a “deviation value”, and thus identifying physiologic vs nonphysiologic use of the PAP device 100, effective or ineffective use of the PAP device 100, or use of the PAP device 100 by a subject with an abnormal medical conditions. The computing device 220 may comprise one or more AI modules 230 configured to apply an artificial neural network or the like, as discussed in embodiments of the disclosure.

In embodiments, the computing device 220 may be configured to obtain one or more parameters recorded by the PAP device 100 via wireless communication, wired communication, or by transfer of a memory from the PAP device 100 to the computing device 220. The computing device 220 may obtain additional parameters from additional sensors or devices. The communication modules 112, 226 of the PAP device 100 and/or the computing device 220 and/or a further device or sensor may include any interface suitable for electronic communication, such as over a short-range wireless network, a long-range wireless network (i.e., mobile data network, internet network, etc.) or a wired connection, such that the computing device 220 is able to transmit and receive information between the PAP device 100 and/or additional sensors or devices. One or more of the PAP device 100 and/or the computing device 220 and/or a further device or sensor may further be arranged to communicate using a restricted or secure connection, such as peer-to-peer communication, an encrypted communication, etc. Further, in some aspects, the PAP device 100 may comprise the computing device 220, such that the PAP device 100 and the computing device 220 may be implemented as effectively comprising a single computing system or being integrated into a single device. In integrated embodiments, the PAP device 100 and the computing device 220 may be operated using the same processor and/or the same memory such that some or all of a method of the current disclosure may be performed locally at the PAP device 100.

The method described herein may be used to detect whether measured signals of the treatment originate from physiologic use of the PAP device by a target subject and may be based on the data provided by the PAP device, effective or ineffective use of the PAP device 100, or use of the PAP device 100 by a subject with an abnormal medical conditions. In another embodiment, such detections may be made with the additional use of a Nox T3 device or another device recording physiological signals such as may be related to respiration, pulse, electrocardiogram, or the like. Furthermore, the method can be used for chain-of-custody purposes by confirming that a PAP device is appropriately used by a target subject.

The subject may be, for example, a human subject, a patient, or an animal. As used herein, such a signal may be termed a signal of the physiological study or treatment. The term “signal” of the physiological study or treatment is used to describe a detection of an event relating to, originating from, or produced by the subject of the physiological study or treatment.

A physiological event may be related to respiration, heartbeat, body movement, brain activity, skin conductance, muscle tone, eye movement, or sound, snoring, response to exterior stimulus or stimuli, such as lighting, sounds, or physical touching, coupled with said exterior stimulus or stimuli, or coupling between physiological events. Additionally, the above categories of physiological events may be further separated into more finely defined events or parameters. For example, respiration may be broken down and considered with respect to respiratory rate, minute ventilation, tidal volume, inspiratory time, pressure, inspiratory pressure, peak inspiratory pressure, and expiratory pressure. The signal of the physiological study may be obtained with one or more biosensors or sensors, such as light sensors, pressure sensors, airflow sensors, temperature sensors, humidity sensors, cameras, accelerometers, microphones, or some other detector configured to gather the signal in the physiological study or treatment.

To improve the quality of the study or treatment, it would be helpful to determine with high accuracy whether the use of the therapeutic device is physiologic for the target subject. Thus, an object of the method and systems disclosed herein is to determine whether the signals obtained from the device represent physiologic use for the target subject. There are multiple scenarios in which this would be useful.

One scenario is that a subject intentionally cheats in use of the therapeutic device. Such a therapeutic device could include a PAP device assigned to a subject for treatment of a condition such as sleep apnea. Sleep apnea is a medical condition where patients routinely have transiently obstructed breathing events during sleep, either completely (apnea), or partially (hypopnea). These apnea and hypopnea events result in blood oxygen desaturation and cortical arousals. Sleep apnea can severely disrupt sleep causing excessive daytime sleepiness and can place an undue burden on the body, increasing the risk of comorbidities such as high blood pressure, cardiac disease, obesity, diabetes, and mental disorders such as depression and anxiety.

Notably, while a PAP device may be physiologically effective in allowing a subject to obtain adequate rest, some subjects may consider the PAP device to be uncomfortable, embarrassing, or may otherwise resent that they are required to use the device. However, the subject may be required to show evidence of using the device, for example in the case of a truck driver with sleep apnea who could lose a driver's license if not complying with prescribed treatments or a military veteran with sleep apnea who could lose disability benefits if not complying with prescribed treatments.

Usually, when a subject is required to show evidence of using a therapeutic device like a PAP device, the PAP device records a log showing operation of the device. While the PAP device may not record usage when the cyclic airflow of breathing is not detected, a subject may develop several different ways to attempt to “cheat” the device, such as by simulating breathing using mechanical means. This can be done by attaching a pump to the PAP device to simulate breathing, and/or by attaching the PAP device to a pressure chamber or bag that flexibly simulates breathing in response to the pressure generated by the PAP device.

Further, in other scenarios, some subjects may innocently misuse a therapeutic device, whether due to improper fit, misunderstanding the device, or because the device is unintentionally dislodged during sleep. Using the method described herein, it becomes possible to verify, or determine, physiologic and nonphysiologicuse in the described scenarios.

One object of this disclosure is to describe how one or more parameters obtained from the signals may be used for automatically determining whether the use of the therapeutic device was or is physiologic for a target subject. Automatically processing, classifying and verifying one or more parameters obtained from a PAP device advantageously provides an effective transformation of measured signals into actionable information for presentation to a clinician and/or directly to a subject. Thus, monitoring of relevant use of the PAP device by a clinician and compliance by a subject are increased, while the required input from a clinician is reduced.

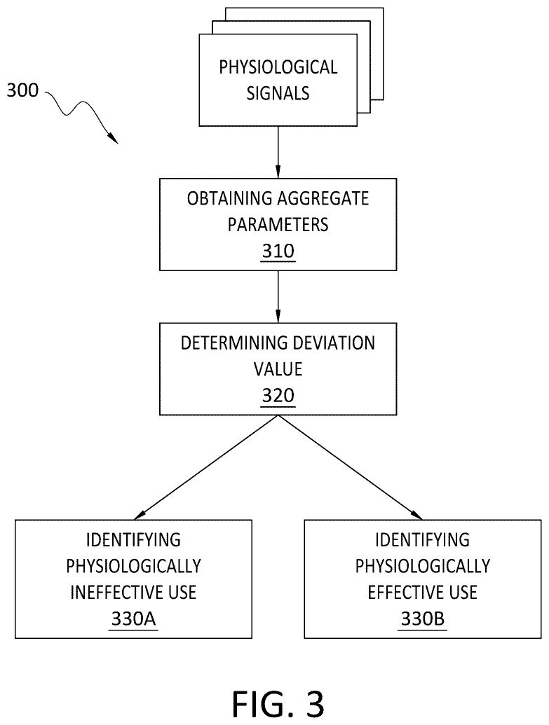

FIG. 3 illustrates a method 300 that may be performed by a computing device or related system, such as described above, for identifying or classifying physiologically effective use of a PAP device. The method 300 may include obtaining 310 one or more parameters recorded by a PAP device, such as from the PAP device or an intervening device, network or memory. A deviation value of the one or more parameters or aggregate parameters may be determined at step 320, and simulated use may be identified 330A when the deviation value of any of the monitored parameters is outside a predefined tolerance range and a physiologic use may be identified 330B when the deviation value is inside the predefined tolerance range.

The step of obtaining 310 the one or more parameters may include recording, compiling, processing and/or storing the one or more parameters. As such, the one or more parameters may comprise averaged values over a predetermined period of time, may include a set of parameters covering a predetermined period of time, may be arranged to include a timestamp, or otherwise modified for communication and evaluation as preferred for the requirements of a given parameter. Similarly, additional information may be added to the parameters for assisting in a processing of the parameters. For example, a device or subject identifier may be included in the one or more parameters, the identifier being associated with a subject or the device and information from the subject's medical history, preferences or concerns identified by the subject or a clinician, and/or privacy requirements related to the subject. In this way a more accurate evaluation of the parameters may be performed, and further information may be provided informing a clinician, the subject or a health monitoring system of appropriate next steps.

In one aspect, a method of the disclosure may comprise obtaining one or more parameters from the PAP device, such as for determining a usage value. For example, using parameters of the PAP device an initial usage value may be determined based on a duration of activity in the PAP device. In other words, an initial usage value may establish a length of time in which the PAP machine appears to have been operational based on a relation between the one or more parameters collected and the corresponding time of collection. The initial usage value may be modified to yield a corrected value that reflects actual use to determine true compliance and physiologic use for the PAP device and target subject.

In varying embodiments, the initial usage value may be modified to consider only operation time of the PAP device where parameters of the PAP device meet predefined conditions. The initial usage value may be modified to form a corrected usage value using parameters of maximum pressure, minimum pressure, and delivered pressure from the PAP device. The corrected usage value may be obtained by taking into account only operation of the PAP device where the range of values of the one or more parameters is inside a predefined tolerance range.

Autotitrating CPAP devices have algorithms to adjust the delivered pressure based on the patient or subject's needs, determined by parameters including consistency of airflow. Typically, the range of pressures delivered over the course of 1 day/night is greater than 1 cm of water pressure. Consequently, it would be unusual for a pressure range to be 1 cm of water or less, which would indicate simulated use of the device, rather than physiologic use as intended by a human. Accordingly, where this range is less than or equal to one, that day of usage may be excluded from the cumulative value, if at least one other parameter is consistent with simulated use.

In another aspect, the range of deviation values for aggregate usage data over more than 1 day may comprise a difference between an upper percentile pressure and a median pressure while the expected range may be defined as greater than one. Accordingly, where the difference between an upper percentile pressure and a median pressure is less than or equal to one, suspicion is raised for simulated use, ineffective use, or use by a subject having an abnormal medical condition.

In varying embodiments, one or more parameters and corresponding predefined tolerance ranges may be defined or employed to determine whether use of the PAP device was physiologic or simulated, ineffective, or whether the PAP device was used by a subject having an abnormal medical condition. Where more than one parameter is employed, a corrected usage value may be obtained by taking into account only operation of the PAP device where at least one deviation value of the one or more parameters is inside a corresponding predefined tolerance range, at least two deviation values, at least three, at least four, at least five, at least six, at least seven, at least eight, at least nine, or at least ten deviation values.

An illustrative embodiment may employ at least two parameters with corresponding predefined tolerance ranges, such as a first deviation value comprising a difference between a maximum pressure and a minimum pressure with a first predefined tolerance range defined as less than or equal to 1 and a second deviation value comprising a difference between an upper percentile pressure and a median pressure with a second predefined tolerance range defined as greater than 1. For ease of understanding, in this example both of the first and the second variance values are required to be inside the corresponding first and second predefined tolerance ranges for the usage of the PAP device to be identified as simulated, not physiologic, or not effective. These conditions may be represented as:

Pressure Settings (to exclude fixed-setting CPAP):

[Maximum Pressure-Minimum Pressure]>1 (1)

### AND

Delivered Pressures (reflecting limited variation of pressure):

[Upper Percentile Pressure-Median Pressure]≤1 (2)

An upper percentile, according to varying aspects of the disclosure, may be a 99^{th} percentile value, a 95^{th} percentile value, a 90^{th} percentile value, an 85^{th} percentile value, an 80^{th} percentile value, a 75^{th} percentile value, or may be a percentile in a range of 75^{th} to 99^{th}, more particularly in a range of 80^{th} to 99^{th}, more particularly in a range of 85^{th} to 95^{th}, or in a range of 90^{th} to 95^{th}.

The parameters obtained by the PAP device may include maximum pressure, minimum pressure, and delivered pressure, as are advantageously already available in many common PAP devices, such that a corrected usage value may be determined without the need for modifications to pre-existing devices. The advantages of the current disclosure may accordingly be realized at low cost and with high efficiency.

The maximum pressure, minimum pressure, delivered pressure (including an upper percentile pressure and a median pressure), and/or other parameters for use in determining a deviation value may be calculated for a predefined period. A predefined period may be determined to correspond to a particular time of day and/or night, or to correspond to a particular length of time. For example, the predefined period may be defined as from 8:00 pm to 8:00 am, or may be defined as a month, a week, a 24 hour period, a 12 hour period, a 10 hour period, an 8 hour period, a four hour period, a two hour period, or the like. In this manner, one or more parameters obtained by the PAP device may be evaluated by calculating maximum pressure, minimum pressure, and delivered pressure (including an upper percentile pressure and a median pressure) monthly, weekly, daily, nightly, and/or in other increments. The predefined period used for determining the deviation values may be selected to be larger to increase confidence in the calculated deviation values and/or may be selected to be smaller for increased precision in detecting the extent of misuse of the PAP device.

Notably, while the maximum pressure, minimum pressure, and delivered pressure are reliable in the detection of consistent use of cheat device, such as a breathing simulator, intermittent use and partial use may be more difficult to detect. Likewise, misuse of a PAP device may be less consistent and more difficult to recognize.

In embodiments, modification of the initial usage value may include identifying non-physiologic deviation values that are outside of a predefined tolerance range and excluding the intervals that contain the nonphysiologic values to determine a corrected usage value. For example, a predefined tolerance range may be defined by the range of human physiology for a corresponding parameter, and parameters falling outside of the corresponding predefined tolerance range may then be excluded from the corrected usage value. In other words, while an initial usage value may merely represent a duration of operation for the PAP device, a corrected usage value may be configured to represent a duration of physiologic operation or use of the PAP device by excluding periods of operation representing simulated use, ineffective use, or use by a subject having an abnormal medical condition, where parameters do not fall within the corresponding predefined tolerance range or ranges. Such identifiable or determinable medical conditions may include, but are not limited to, abnormally high respiratory rate, low tidal volume, low minute ventilation of the subject.

The parameters considered may include, but are not limited to, one or more of respiratory rate, minute ventilation, tidal volume, inspiratory time, pressure, inspiratory pressure, peak inspiratory pressure, and expiratory pressure. These parameters may be collected by the PAP device alone, or by a combination of the PAP device and an additional sensor or sensors.

In an example, a combination of delivered pressure, respiratory rate, tidal volume, and minute ventilation may be used as conditions for identifying non-physiologic use of a PAP device from obtained one or more parameters. The predefined tolerance ranges for each of the parameters may be defined by a typical normal range according to the table below:

| | Typical Normal | | Range limits |
|---|---|---|---|
| | Range | | for detection |
| [95th Percentile Pressure] − | x > 1 | | x < 1 |
| [Median Pressure] | | | |
| Respiratory rate (resting): | 12-16/min | | x < 8 or x > 30 |
| Tidal Volume (resting) | 400-600 | ml | x < 400 ml or |
| | | | x > 800 ml |
| Minute Ventilation (resting) | 5-8 | L | x < 4 L or x > 10 L |

In this example, usage intervals containing parameters falling outside of the typical normal range of detection may be excluded from a corrected usage value. Of course, a corrected usage value may be determined using one or more of maximum pressure, minimum pressure, delivered pressure, respiratory rate, minute ventilation, tidal volume, inspiratory time, pressure, inspiratory pressure, peak inspiratory pressure, expiratory pressure, and other parameters, in varying embodiments of the disclosure.

In another embodiment, a leak value or leak rate is a considered or analyzed parameter, particularly in determining an effective or ineffective use of the PAP system. In general, a leak rate of 0.4 liter per second (24 liters per minute) may be considered acceptable. A higher volume of leak is associated with patient discomfort, interrupted sleep and suboptimal efficacy of the PAP. As the pressure in the CPAP system increases the leak volume increases as well. However, if pressure does not increase but leak occurs then effective pressure will drop. Data or signals relating to the leak value or leak rate may be monitored and deviation values may be determined when the leak rate falls outside a predetermined or expected range, such as, for example, a leak rate of greater than 0.4 liters per second, greater than 0.5 liters per second, greater than 0.6 liters per second, or 0.7 liters per second.

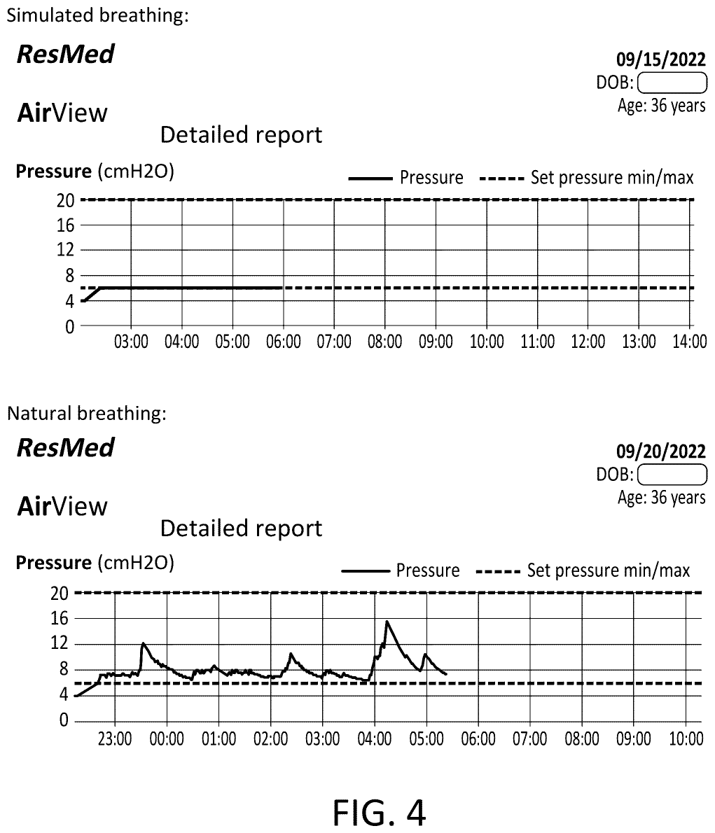

In an aspect of the current disclosure, non-physiologic values can be distinguished by a magnitude of respective parameters as described above and, advantageously, also by consistency of the parameters within a predefined period of use. In one aspect, physiologic use or ineffective use of a PAP device may be determined based on expected variability of the values of one or more parameters from the PAP device for one or more parameters that are dependent on physiologic and/or pathophysiologic factors. For example, respiratory rate, minute ventilation, tidal volume, inspiratory time, pressure, inspiratory pressure, and expiratory pressure are all parameters that should have a minimum level of variability. This variability may be better understood with reference to FIG. 4, which provides a comparison between parameters for simulated breathing and human breathing of a patient treated for obstructive sleep apnea with CPAP.

Accordingly, a deviation value may comprise a difference between a mean and a standard deviation of one or more one or more parameters, a difference between a median and an upper percentile value of one or more one or more parameters, an upper percentile value of one or more parameters, and/or another measure of variance. Modification of an initial usage value may then include identifying intervals with non-physiologic or ineffective values outside of a predefined tolerance range and excluding those intervals to determine a corrected usage value. For example, a predefined tolerance range may be defined by an expected range of values and any values falling outside of the corresponding predefined tolerance range may then be excluded to yield a usage value.

One or more of the above described deviation values may be employed according to methods and systems of the current disclosure. Accordingly, a plurality of values may be evaluated within the context of the expected physiologic ranges for one or more of the parameters described herein, and identification of simulated use of the PAP device may require that at least one of the values is a deviation value falling outside a corresponding predefined physiologic tolerance range. In this aspect, a first deviation value may correspond to a first parameter and a second deviation value may correspond to a second parameter, up to an nth parameter. Misuse of a PAP device may be detected with increased accuracy due to the use of an increased number of parameters, advantageously ensuring that simulated or ineffective use of the device may be identified and remedied even where one or more parameters may otherwise independently appear to correspond to physiologic use of the device.

In another aspect, values may themselves be compared across predefined periods for finding a level of difference between the predefined periods, such as night-to-night or the like, as discussed above.

In varying embodiments of the disclosure, one or more parameters may comprise time series data, for example, a recorded flow of the PAP device, and a deviation value for each corresponding parameter from a predefined tolerance range. These may comprise one or more of an amplitude of the time series, a frequency of the time series, frequency components of the time series, and/or ratios of an amplitude of a first frequency band and a second frequency band. Modification of an initial usage value may then include identifying non-physiologic, deviation values for each parameter, if it is outside of a predefined tolerance range, and excluding intervals containing such deviation values to determine a corrected usage value.

Some embodiments may determine predefined tolerance ranges based on a target subject. For example, a typical normal range of detection may be unique to the physiology of the target subject, which may be determined based on recorded parameters from the target subject's previous use of a PAP machine, recorded parameters from a sleep study of the target subject, or projected parameters based on physical characteristics of the target subject.

In an embodiment, predefined tolerance ranges for one or more parameters may be determined based on a “sleep fingerprint” of a target subject, the specific physiologic characteristics of that subject. The sleep fingerprint of the target subject may be obtained by conducting a sleep study to determine a typical normal range of detection for one or more parameters, for example, using Nox Medical's T3 device or another similar sleep study device. Based on the sleep fingerprint derived from the sleep study, the predefined tolerance ranges used for evaluating use of the PAP device may be made specific to the target subject, advantageously increasing accuracy for evaluating use of the device and allowing to determine that the target subject was indeed the source of the one or more parameters recorded by the PAP device.

With use of a sleep fingerprint as described above, the sleep fingerprint may be static or may be dynamically determined. For example, while a sleep fingerprint may be based on a limited sleep study, a sleep fingerprint may be dynamically updated during use of the PAP device, whether by continued use of Nox Medical's T3 device or another similar sleep study device in conjunction with the PAP device, or by updating the sleep study based on parameters recorded by the PAP device during physiologic or effective use of the device. The use of multiple sensors or devices, and particularly redundant sensors, advantageously enables automatic verification of the measurements collected by the devices and improves both the speed and accuracy of reported measurements relative to conventional systems. Redundant measurements may be combined or otherwise processed together.

Various features of the disclosure may be better understood by reference to specific examples of a method for identifying physiologic or effective use of a PAP device according to the current disclosure, as detailed below. The examples provided are illustrative in nature of a single application of principles according to the disclosure and are not intended to be limiting.

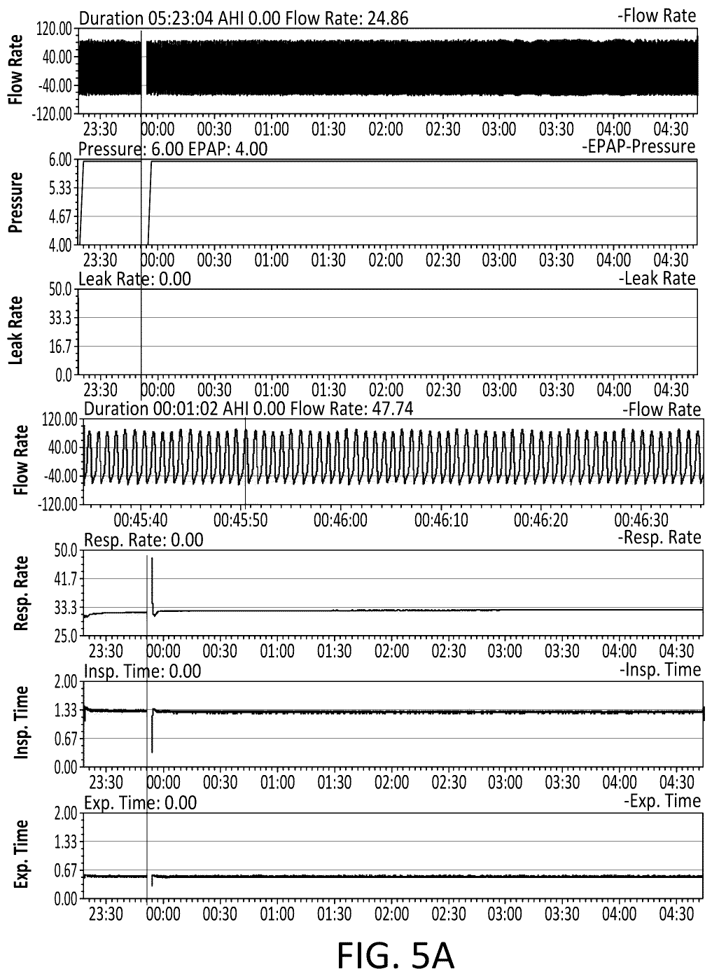

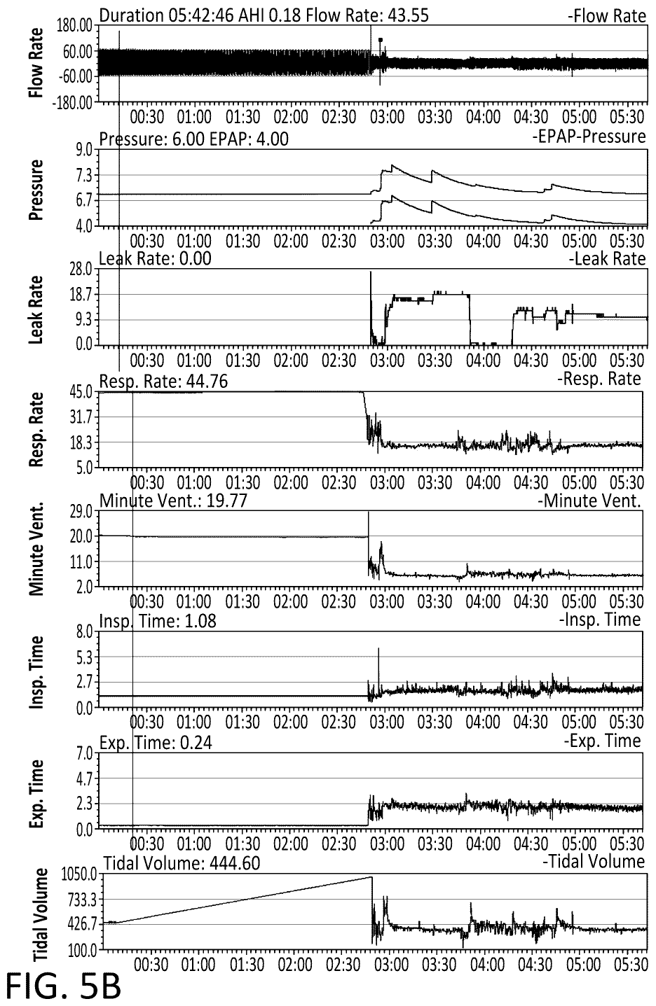

Example 1: FIGS. 5A-5B include examples of one or more parameters obtained from a PAP device of a target subject where use of the PAP device was simulated by a mechanical device that produced cyclic airflow. FIGS. 5A-5B provide data obtained from a memory card of the PAP device. Notably, certain parameters show nonphysiologic consistency of values throughout the period of use, lacking the expected physiologic variation, as well as clearly nonphysiologically high flow rate, much higher than a normal human respiratory rate.

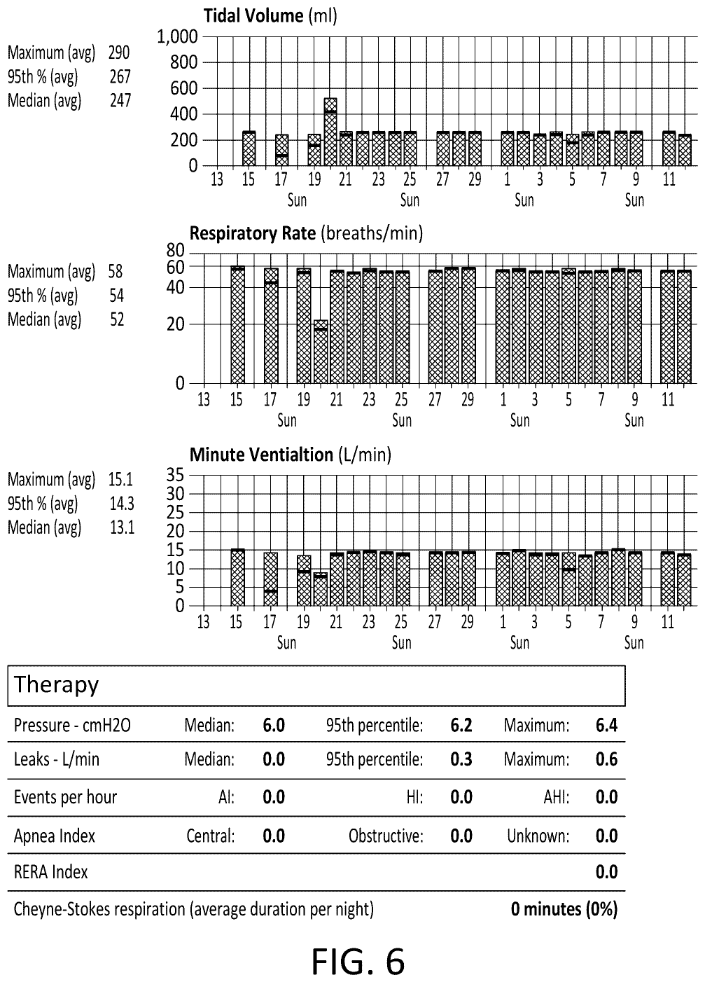

Example 2: FIG. 6 includes examples of one or more parameters obtained from a PAP device of another target subject where use of the PAP device was simulated by a device generating cyclic airflow. According to the method of the current disclosure, suspicion was raised by the difference between the 95^{th} percentile pressure and the median pressure being less than or equal to one. Using the described embodiments, the one or more parameters of FIG. 6 are found to include an abnormally low tidal volume but a respiratory rate more than three times higher than normal, resulting in abnormally high minute ventilation (with the exception of 9/20, when the target subject's use of the PAP device was physiologic).

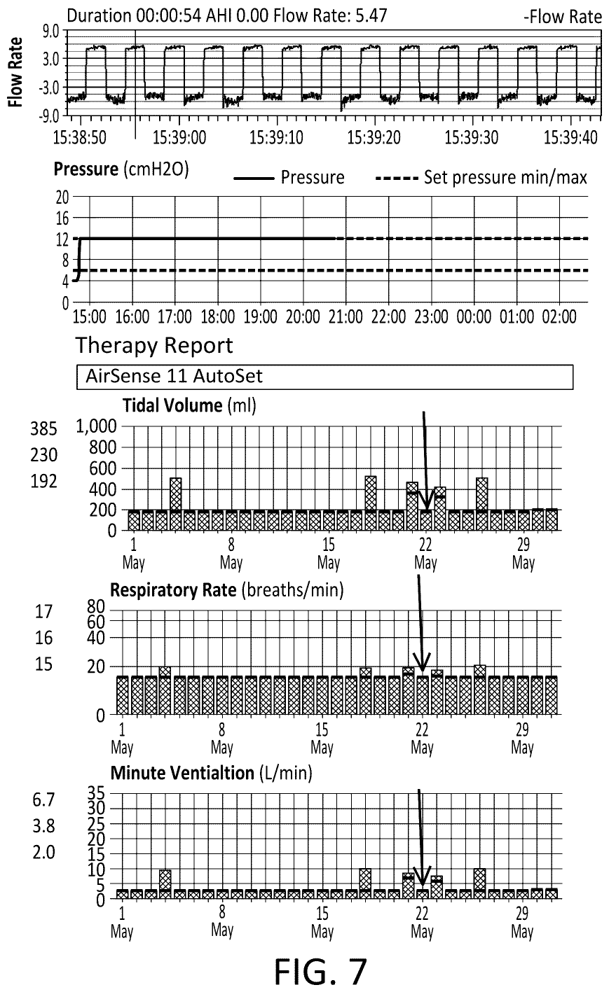

Example 3: FIG. 7 includes examples of one or more parameters obtained from a PAP device of another target subject with normal respiratory rate simulation but abnormally low tidal volume and minute ventilation. Furthermore, when considering expected normal day-to-day variability of the values, the method of the disclosure indicates that the parameters are exceptionally consistent and correspond to a simulated use of the PAP device.

The embodiments of the present disclosure may advantageously facilitate regular or real-time monitoring by automatically processing, classifying and verifying measurements recorded from a target subject's body, and performing dynamic actions based thereon. Automatically processing, classifying and verifying measurements advantageously provides an effective filtering of real-time data into actionable information for presentation to a clinician and/or directly to a subject. Thus, monitoring of relevant measurements by a clinician and compliance by a user may be increased, while the required input from a clinician may be reduced.

The computing device 220 may be arranged to automatically evaluate and classify the one or more parameters obtained from the PAP device, for example as physiologic use or simulated use. A flag or other identifier may be assigned to the parameters or a time period to which they correspond on the basis of the classification, such that the one or more parameters may be stored with or otherwise associated with said classification. The computing device 220 may further output the one or more parameters and corresponding flag as a readable image, document, or message. For example, where use of the PAP device is flagged as being simulated, the simulated data can be excluded from the tabulation of valid compliance with the treatment, and a message may be sent to notify a clinician or the subject themselves.

In some embodiments the clinician may review one or more parameters for verification purposes where use of a PAP device is flagged as being simulated. In a further aspect of the disclosure, verification by a clinician may be provided to the computing device 220 as a reference for updating the predefined tolerance ranges and/or training a learning artificial neural network, as necessary, such that the accuracy of the classification by the computing device 220 may increase with use, further reducing the intervention required from a reviewer or clinician.

The method and related systems and devices according to the disclosure advantageously improve the functionality of a computer system on or in cooperation with which the method or automated method is carried out by providing specific rules allowing for automatic evaluation and action in response to the use of a PAP device by a user, reducing the processing requirements of the system while improving the efficiency and capabilities of remote monitoring.

In embodiments, the method of identifying a physiologic or ineffective use of a PAP device may be performed with machine learning, such as using a learning artificial neural network or the like for conducting an evaluation of the one or more parameters recorded by the PAP device. Such a neural network or machine learning algorithm may adjust and improve tolerance ranges for values through automated learning and/or training, for example using training datasets. In embodiments of the method and related systems and devices, sensors related to a PAP device, and related to relevant parameters, and a learning artificial neural network architecture may be synergistically combined to classify physiologic or simulated or ineffective use of a PAP device.

Embodiments of the present disclosure may comprise or utilize a special-purpose or general-purpose computer system that includes computer hardware, such as, for example, one or more processors and system memory, as discussed in greater detail below. Embodiments within the scope of the present disclosure also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, embodiments of the disclosure can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” may be defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions may comprise, for example, instructions and data which, when executed by one or more processors, cause a general-purpose computer system, special-purpose computer system, or special-purpose processing device to perform a certain function or group of functions. Computer-executable instructions may be, for example, binaries, intermediate format instructions such as assembly language, or even source code.

The disclosure of the present application may be practiced in network computing environments with many types of computer system configurations, including, but not limited to, personal computers, desktop computers, laptop computers, message processors, hand-held devices, multi-processor systems, microprocessor-based or programmable consumer electronics, network PCs, minicomputers, mainframe computers, mobile telephones, PDAS, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The disclosure of the present application may also be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

By providing a method and related systems and devices for identifying physiologic vs simulated use of a PAP device or effective vs ineffective use of the PAP device according to disclosed embodiments, the problem of misuse or ineffective use of PAP devices is addressed. The disclosed embodiments advantageously provide a method and related systems and devices that identifies and provides increased accuracy, speed and consistency in distinguishing between physiologic and simulated use or effective or ineffective use of a PAP device in order to provide actionable and quantifiable insights to a clinician or automated system.

Not necessarily all such objects or advantages may be achieved under any embodiment of the disclosure. This disclosure may be embodied or carried out to achieve or optimize one advantage or group of advantages as taught without achieving other objects or advantages as taught or suggested. Further, various steps and/or components from different embodiments described may be interchangeable. Besides the variations described, other equivalents for each feature can be mixed and matched to construct or use a method and related systems and devices for identifying physiologic vs simulated use and ineffective vs effective use of a PAP device under principles of the present disclosure.

Also according to the present disclosure, a method for maintaining a chain of custody of physiological study data for a subject is provided, comprising any of the above-described methods or combinations or permutations of the above-described methods, including at least the following the method comprising: providing to the subject a physiological study system, the physiological study system including one or more sensors configured to obtain physiological study data, the physiological study data including a first signal from the physiological study and a second signal from the physiological study; obtaining a confirmation that the one or more sensors have been placed on the subject; receiving the physiological study data; extracting a first signal from the physiological study; extracting a second signal from the physiological study; determining a coherency value between components of the extracted first signal and components of the extracted second signal; and determining the correspondence of the physiological study data based on the determined coherency value. The physiological study may be a sleep study.

Certain terms are used herein throughout the description and claims to refer to particular methods, features, or components. Different persons may refer to the same methods, features, or components by different names. This disclosure does not intend to distinguish between methods, features, or components that differ in name but not function. The figures are not necessarily to scale. Certain features and components herein may be shown in exaggerated scale or in somewhat schematic form and some details of conventional elements may not be shown or described in interest of clarity and conciseness.

Although various example embodiments have been described in detail herein, many modifications are possible in the example embodiments without materially departing from the concepts of present disclosure. Accordingly, any such modifications are intended to be included in the scope of this disclosure. Likewise, while the disclosure herein contains many specifics, these specifics should not be construed as limiting the scope of the disclosure or of any of the appended claims, but merely as providing information pertinent to one or more specific embodiments that may fall within the scope of the disclosure and the appended claims. Any described features from the various embodiments disclosed may be employed in combination. In addition, other embodiments of the present disclosure may also be devised which lie within the scopes of the disclosure and the appended claims. Each addition, deletion, and modification to the embodiments that falls within the meaning and scope of the claims is to be embraced by the claims.

The various features disclosed in the present disclosure are interchangeable. Besides the variations described, other known equivalents for various features can be mixed and matched to make methods, apparatus, and systems for determining a physiological effectiveness of airway pressure devices under principles of the present disclosure.

Although this disclosure describes certain exemplary embodiments and examples of methods, apparatus, and systems for determining physiological effectiveness of airway pressure devices, the present disclosure extends beyond the specifically disclosed embodiments to other alternative embodiments and/or uses of the disclosure and modifications and equivalents thereof. It is intended that the present disclosure should not be limited by the particular disclosed embodiments described above.

The disclosure further relates to several embodiments as identified by the below numbered embodiments. The embodiments are provided only to demonstrate non-limiting examples of possible embodiments of the disclosure.
1-1. A method for determining a physiologic, vs simulated use of a positive airway pressure machine, the method comprising: obtaining one or more parameters recorded by the positive airway pressure machine; determining whether one or more parameters are within expected physiologic range for each parameter; identifying simulated use when a deviation value is determined from said values to be outside the predefined tolerance range; and identifying physiologic use when the all of said values or a predetermined number of said values are inside the predefined tolerance ranges.
1-2. The method according to any or a combination of one or more of 1-1 above and 1-3 to 1-16 below, wherein the one or more parameters comprise one or more of maximum pressure, minimum pressure, delivered pressure, respiratory rate, minute ventilation, tidal volume, inspiratory time, inspiratory pressure, peak inspiratory pressure, and expiratory pressure.
1-3. The method according to any or a combination of one or more of 1-1 to 1-2 above and 1-4 to 1-16 below, wherein the one or more parameters comprise two or more of maximum pressure, minimum pressure, delivered pressure, respiratory rate, minute ventilation, tidal volume, inspiratory time, inspiratory pressure, peak inspiratory pressure, and expiratory pressure.
1-4. The method according to any or a combination of one or more of 1-1 to 1-3 above and 1-5 to 1-16 below, wherein the deviation value comprises at least a first deviation value and a second deviation value, and wherein the predefined tolerance range comprises at least a first predefined tolerance range corresponding to the first deviation value and a second predefined tolerance range corresponding to the second deviation value.
1-5. The method according to any or a combination of one or more of 1-1 to 1-4 above and 1-6 to 1-16 below, wherein the step of identifying simulated use when the deviation value is outside a predefined tolerance range comprises identifying a simulated use when one or more of at least the first deviation value and the second deviation value are outside of the corresponding first predefined tolerance range and second predefined tolerance range.
1-6. The method according to any or a combination of one or more of 1-1 to 1-5 above and 1-7 to 1-16 below, wherein the deviation value comprises a difference between a mean and a standard deviation of the one or more parameters.
1-7. The method according to any or a combination of one or more of 1-1 to 1-6 above and 1-8 to 1-16 below, wherein the deviation value comprises a difference between a median and an upper percentile value of the one or more parameters.
1-8. The method according to any or a combination of one or more of 1-1 to 1-7 above and 1-9 to 1-16 below, wherein the upper percentile value comprises a 90^{th} percentile value.
1-9. The method according to any or a combination of one or more of 1-1 to 1-8 above and 1-10 to 1-16 below, wherein the upper percentile value comprises a 95^{th} percentile value.
1-10. The method according to any or a combination of one or more of 1-1 to 1-9above and 1-11 to 1-16 below, wherein the deviation value comprises an upper percentile value in a range of an 80^{th} percentile to a 99^{th} percentile.
1-11. The method according to any or a combination of 1-1 to 1-10 above and 1-12 to 1-16 below, wherein the one or more parameters comprise time series data.
1-12. The method according to any or a combination of one or more of 1-1 to 1-11above and 1-13 to 1-16 below, further comprising: determining an initial usage value based on the one or more parameters, and determining a corrected usage value taking into account said identifying physiologic use and said identifying the simulated use.
1-13. The method according to any or a combination of one or more of 1-1 to 1-12above and 1-14 to 1-16 below, further comprising: collecting one or more parameters using the positive airway pressure machine.
1-14. The method according to any or a combination of one or more of 1-1 to 1-13above and 1-15 to 1-16 below, further comprising: determining the predefined tolerance range based on a sleep fingerprint of a target subject.
1-15. The method according to any or a combination of one or more of 1-1 to 1-14above and 1-16 below, further comprising: determining whether the one or more parameters correspond to a “sleep fingerprint” of a target subject.
1-16. The method according to any or a combination of one or more of 1-1 to 1-15above, wherein at least two or more parameters are assessed for values that are outside the corresponding tolerance ranges, wherein the step of identifying a simulated use when one or more of the at least two or more parameters have values outside the corresponding predefined tolerance ranges, and wherein the step of identifying a physiologic use when each of the at least two or more parameters have values inside of the corresponding predefined tolerance ranges.
1-17. A system configured to determine a physiologic vs simulated use of a positive airway pressure machine, the system comprising: one or more processors; and one or more memory storages, wherein the one or more memory storages have stored thereon, or the system is configured to receive, one or more parameters obtained by the positive airway pressure machine, wherein the one or more processors are configured to perform the following: determine values for the one or more parameters; identify a simulated use when a deviation value is determined from said values to be outside a predefined tolerance range, and identify a physiologic use when the value is inside the predefined tolerance range.
1-18. The system according to any or a combination of one or more of 1-17 above and 1-19 to 1-20 below, wherein the one or more parameters comprise one or more of maximum pressure, minimum pressure, delivered pressure, respiratory rate, minute ventilation, tidal volume, inspiratory time, inspiratory pressure, peak inspiratory pressure, and expiratory pressure.
1-19. The system according to any or a combination of one or more of 1-17 to 1-18above and 1-20 below, further comprising the positive airway pressure machine, the positive airway pressure machine configured to record the one or more parameters using one or more sensors of the positive airway pressure machine and to communicate the one or more parameters to the one or more processors.
1-20. The system according to any or a combination of one or more of 1-17 to 1-19above, further comprising a sleep study device configured to record a sleep fingerprint of a target subject and communicate the sleep fingerprint to the one or more processors.
1-21. A system configured to determine effectiveness of use of a positive airway pressure machine, the system comprising: one or more processors; and one or more memory storages, wherein the one or more memory storages have stored thereon, or the system is configured to receive, one or more parameters obtained by the positive airway pressure machine, wherein the one or more memory storages have stored thereon executable instructions that when executed by the one or more processors configure the one or more processors to perform the method of any or combination of one or more of 1-1 to 1-16 above.
1-22. One or more computer-readable mediums having stored thereon executable instructions that when executed by the one or more processors configure a computer system to perform at least the following: obtain one or more parameters recorded by a positive airway pressure machine; determine values for the one or more parameters; identify a simulated use when the deviation value is determined from said values to be outside a predefined tolerance range, and identify a physiological use when the variance value is inside the predefined tolerance range.
1-23. One or more computer-readable mediums having stored thereon executable instructions that when executed by the one or more processors configure a computer system to perform the method of any or combination of one or more of 1-1 to 1-16 above.

The disclosure also further relates to several embodiments as identified by the below numbered embodiments. The embodiments are provided only to demonstrate non-limiting examples of possible embodiments of the disclosure.
2-1. A method for determining effectiveness of use of a positive airway pressure machine, the method comprising: obtaining one or more parameters recorded by the positive airway pressure machine; determining whether one or more parameters are within expected physiologic range for each parameter; identifying ineffective use when a deviation value is determined from said values to be outside the predefined tolerance range; and identifying effective use when the all said values or a predetermined number of said values are inside the predefined tolerance ranges.
2-2. The method according to any or a combination of one or more of 2-1 above and 2-3 to 2-16 below, wherein the one or more parameters comprise one or more of maximum pressure, minimum pressure, delivered pressure, respiratory rate, minute ventilation, tidal volume, inspiratory time, inspiratory pressure, peak inspiratory pressure, and expiratory pressure.
2-3. The method according to any or a combination of one or more of 2-1 to 2-2 above and 2-4 to 2-16 below, wherein the one or more parameters comprise two or more of maximum pressure, minimum pressure, delivered pressure, respiratory rate, minute ventilation, tidal volume, inspiratory time, inspiratory pressure, peak inspiratory pressure, and expiratory pressure.
2-4. The method according to any or a combination of one or more of 2-1 to 2-3 above and 2-5 to 2-16 below, wherein the deviation value comprises at least a first deviation value and a second deviation value, and wherein the predefined tolerance range comprises at least a first predefined tolerance range corresponding to the first deviation value and a second predefined tolerance range corresponding to the second deviation value.
2-5. The method according to any or a combination of one or more of 2-1 to 2-4 above and 2-6 to 2-16 below, wherein the step of identifying an inefficient use when the deviation value is outside a predefined tolerance range comprises identifying a inefficient use when one or more of at least the first deviation value and the second deviation value are outside of the corresponding first predefined tolerance range and second predefined tolerance range.
2-6. The method according to any or a combination of one or more of 2-1 to 2-5 above and 2-7 to 2-16 below, wherein the deviation value comprises a difference between a mean and a standard deviation of the one or more parameters.
2-7. The method according to any or a combination of one or more of 2-1 to 2-6 above and 2-8 to 2-16 below, wherein the deviation value comprises a difference between a median and an upper percentile value of the one or more parameters.
2-8. The method according to any or a combination of one or more of 2-1 to 2-7 above and 2-9 to 2-16 below, wherein the upper percentile value comprises a 90^{th} percentile value.
2-9. The method according to any or a combination of one or more of 2-1 to 2-8 above and 2-10 to 2-16 below, wherein the upper percentile value comprises a 95^{th} percentile value.
2-10. The method according to any or a combination of one or more of 2-1 to 2-9above and 2-11 to 2-16 below, wherein the deviation value comprises an upper percentile value in a range of an 80^{th} percentile to a 99^{th} percentile.
2-11. The method according to any or a combination of 2-1 to 2-10 above and 2-12 to 2-2 to 2-16 below, wherein the one or more parameters comprise time series data.
2-12. The method according to any or a combination of one or more of 2-1 to 2-11above and 2-13 to 2-16 below, further comprising: determining an initial usage value based on the one or more parameters; and determining a corrected usage value taking into account said identifying effective use and said identifying the ineffective use.
2-13. The method according to any or a combination of one or more of 2-1 to 2-12above and 2-14 to 2-16 below, further comprising: collecting one or more parameters using the positive airway pressure machine.
2-14. The method according to any or a combination of one or more of 2-1 to 2-13above and 2-15 to 2-16 below, further comprising: determining the predefined tolerance range based on a sleep fingerprint of a target subject.
2-15. The method according to any or a combination of one or more of 2-1 to 2-14above and 2-16 below, further comprising: determining whether the one or more parameters correspond to a “sleep fingerprint” of a target subject.
2-16. The method according to any or a combination of one or more of 2-1 to 2-15above, wherein at least two or more parameters are assessed for values that are outside the corresponding tolerance ranges, wherein the step of identifying an ineffective use when one or more of the at least two or more parameters have values outside the corresponding predefined tolerance ranges, and wherein the step of identifying an effective use when each of the at least two or more parameters have values inside of the corresponding predefined tolerance ranges.
2-17. A system configured to determine effectiveness of use of a positive airway pressure machine, the system comprising: one or more processors; and one or more memory storages, wherein the one or more memory storages have stored thereon, or the system is configured to receive, one or more parameters obtained by the positive airway pressure machine, wherein the one or more processors are configured to perform the following: determine values for the one or more parameters; identify an ineffective use when a deviation value is determined from said values to be outside a predefined tolerance range, and identify an effective use when the value is inside the predefined tolerance range.
2-18. The system according to any or a combination of one or more of 2-17 above and 2-19 to 2-20 below, wherein the one or more parameters comprise one or more of maximum pressure, minimum pressure, delivered pressure, respiratory rate, minute ventilation, tidal volume, inspiratory time, inspiratory pressure, peak inspiratory pressure, and expiratory pressure.
2-19. The system according to any or a combination of one or more of 2-17 to 2-18above and 2-20 below, further comprising the positive airway pressure machine, the positive airway pressure machine configured to record the one or more parameters using one or more sensors of the positive airway pressure machine and to communicate the one or more parameters to the one or more processors.
2-20. The system according to any or a combination of one or more of 2-17 to 2-19above, further comprising a sleep study device configured to record a sleep fingerprint of a target subject and communicate the sleep fingerprint to the one or more processors.
2-21. A system configured to determine effectiveness of use of a positive airway pressure machine, the system comprising: one or more processors; and one or more memory storages, wherein the one or more memory storages have stored thereon, or the system is configured to receive, one or more parameters obtained by the positive airway pressure machine, wherein the one or more memory storages have stored thereon executable instructions that when executed by the one or more processors configure the one or more processors to perform the method of any or a combination of one or more of 2-1 to 2-16 above.
2-22. One or more computer-readable mediums having stored thereon executable instructions that when executed by the one or more processors configure a computer system to perform at least the following: obtain one or more parameters recorded by a positive airway pressure machine; determine values for the one or more parameters; identify an ineffective use when the deviation value is determined from said values to be outside a predefined tolerance range, and identify an effective use when the variance value is inside the predefined tolerance range.
2-23. One or more computer-readable mediums having stored thereon executable instructions that when executed by the one or more processors configure a computer system to perform the method of any or combination of one or more of 2-1 to 2-16 above.
2-24. A method for identifying an abnormal medical condition of a subject using a positive airway pressure machine, the method comprising: obtaining one or more parameters recorded by the positive airway pressure machine; determining whether one or more parameters are within expected physiologic range for each parameter; identifying an abnormal medical condition when a deviation value is determined from said values to be outside the predefined tolerance range.

Certain embodiments and features may have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges may appear in one or more claims below. Any numerical value is “about” or “approximately” the indicated value, and takes into account experimental error and variations that would be expected by a person having ordinary skill in the art.

What is claimed:

1. A method for determining a physiological effectiveness of a positive airway pressure device or determining a physiologic versus a simulated use of a positive airway pressure device, the method comprising:
obtaining one or more parameters recorded by the positive airway pressure machine;
determining whether one or more parameters are within expected physiologic range for each parameter;
identifying simulated use when a deviation value is determined from said values to be outside the predefined tolerance range, and
identifying physiologic use when the all of said values or a predetermined number of said values are inside the predefined tolerance ranges.

2. The method according to claim 1, wherein the one or more parameters comprise one or more of maximum pressure, minimum pressure, delivered pressure, respiratory rate, minute ventilation, tidal volume, inspiratory time, inspiratory pressure, peak inspiratory pressure, and expiratory pressure.

3. The method according to claim 1, wherein the one or more parameters comprise two or more of maximum pressure, minimum pressure, delivered pressure, respiratory rate, minute ventilation, tidal volume, inspiratory time, inspiratory pressure, peak inspiratory pressure, and expiratory pressure.

4. The method according to claim 1, wherein the deviation value comprises at least a first deviation value and a second deviation value, and
wherein the predefined tolerance range comprises at least a first predefined tolerance range corresponding to the first deviation value and a second predefined tolerance range corresponding to the second deviation value.

5. The method according to claim 1, wherein the step of identifying simulated use when the deviation value is outside a predefined tolerance range comprises identifying a simulated use when one or more of at least the first deviation value and the second deviation value are outside of the corresponding first predefined tolerance range and second predefined tolerance range.

6. The method according to claim 1, wherein the deviation value comprises a difference between a mean and a standard deviation of the one or more parameters.

7. The method according to claim 1, wherein the deviation value comprises a difference between a median and an upper percentile value of the one or more parameters.

8. The method according to claim 1, wherein the upper percentile value comprises a 90^{th} percentile value.

9. The method according to claim 1, wherein the upper percentile value comprises a 95^{th} percentile value.

10. The method according to claim 1, wherein the deviation value comprises an upper percentile value in a range of an 80^{th} percentile to a 99^{th} percentile.

11. The method according to claim 1, wherein the one or more parameters comprise time series data.

12. The method according to claim 1, further comprising:
determining an initial usage value based on the one or more parameters, and
determining a corrected usage value taking into account said identifying physiologic use and said identifying the simulated use.

13. The method according to claim 1, further comprising:
collecting one or more parameters using the positive airway pressure machine.

14. The method according to claim 1, further comprising:
determining the predefined tolerance range based on a sleep fingerprint of a target subject.

15. The method according to claim 1, further comprising:
determining whether the one or more parameters correspond to a “sleep fingerprint” of a target subject.

16. The method according to claim 1, wherein at least two or more parameters are assessed for values that are outside the corresponding tolerance ranges,
wherein the step of identifying a simulated use when one or more of the at least two or more parameters have values outside the corresponding predefined tolerance ranges, and
wherein the step of identifying a physiologic use when each of the at least two or more parameters have values inside of the corresponding predefined tolerance ranges.

17. A system configured to determine a physiological effectiveness of a positive airway pressure device or determining a physiologic versus a simulated use of a positive airway pressure device, the system comprising:
one or more processors; and
one or more memory storages, wherein the one or more memory storages have stored thereon, or the system is configured to receive, one or more parameters obtained by the positive airway pressure machine,
wherein the one or more processors are configured to perform the following:
determine values for the one or more parameters;
identify a simulated use when a deviation value is determined from said values to be outside a predefined tolerance range, and
identify a physiologic use when the value is inside the predefined tolerance range.

18. The system according to claim 17, wherein the one or more parameters comprise one or more of maximum pressure, minimum pressure, delivered pressure, respiratory rate, minute ventilation, tidal volume, inspiratory time, inspiratory pressure, peak inspiratory pressure, and expiratory pressure.

19. The system according to claim 17, further comprising the positive airway pressure machine, the positive airway pressure machine configured to record the one or more parameters using one or more sensors of the positive airway pressure machine and to communicate the one or more parameters to the one or more processors.

20. The system according to claim 17, further comprising a sleep study device configured to record a sleep fingerprint of a target subject and communicate the sleep fingerprint to the one or more processors.

21. A system configured to determine an effectiveness of use of a positive airway pressure machine, the system comprising:
one or more processors; and
one or more memory storages, wherein the one or more memory storages have stored thereon, or the system is configured to receive, one or more parameters obtained by the positive airway pressure machine,
wherein the one or more memory storages have stored thereon executable instructions that when executed by the one or more processors configure the one or more processors to perform the method according to claim 1.

22. One or more computer-readable mediums having stored thereon executable instructions that when executed by the one or more processors configure a computer system to perform the method according to claim 1.

23. A method for determining an effectiveness of use of a positive airway pressure machine, the method comprising:
obtaining one or more parameters recorded by the positive airway pressure machine;
determining whether one or more parameters are within expected physiologic range for each parameter;
identifying ineffective use when a deviation value is determined from said values to be outside the predefined tolerance range, and
identifying effective use when the all said values or a predetermined number of said values are inside the predefined tolerance ranges.

24. A system configured to perform the method according to claim 23 for determining an effectiveness of use of a positive airway pressure machine, the system comprising:
one or more processors; and
one or more memory storages, wherein the one or more memory storages have stored thereon, or the system is configured to receive, one or more parameters obtained by the positive airway pressure machine,
wherein the one or more processors are configured to perform the following:
determine values for the one or more parameters;
identify an ineffective use when a deviation value is determined from said values to be outside a predefined tolerance range, and
identify an effective use when the value is inside the predefined tolerance range.
